(19) United States
(12) Patent Application Publication
(10) Pub. No.: US 2014/0257257 A1
(43) Pub. Date: Sep. 11, 2014

(54) SYSTEMS AND METHODS FOR TREATING TARGET TISSUE IN THE VITREOUS CAVITY

(71) Applicants: Robert Edward Grant, Laguna Beach, CA (US); Gwillem Mosedale, Muenchen (DE); David Mordaunt, Los Gatos, CA (US)

(72) Inventors: Robert Edward Grant, Laguna Beach, CA (US); Gwillem Mosedale, Muenchen (DE); David Mordaunt, Los Gatos, CA (US)

(21) Appl. No.: 13/793,844

(22) Filed: Mar. 11, 2013

(51) Int. Cl. A61F 9/007; A61F 9/008

(52) U.S. Cl. CPC A61F 9/00709; A61F 9/00802
USPC 606/4

(57) ABSTRACT

A system and its method for treating targeted tissue in the vitreous cavity of an eye include a laser unit for generating a laser beam and a detector for creating an image of the targeted tissue. The system also includes a computer which defines a focal spot path for emulsifying the targeted tissue. A comparator that is connected with the computer then controls the laser unit to move the focal spot of the laser beam. This focal spot movement is accomplished to treat the targeted tissue, while minimizing deviations of the focal spot from the defined focal spot path.

### FIELD OF THE INVENTION

The present invention pertains generally to systems and methods for performing ophthalmic surgical procedures using laser devices. More particularly, the present invention pertains to systems and methods for treating target tissue in the vitreous cavity of an eye. The present invention is particularly, but not exclusively, useful as a system and method for using an OCT guided femtosecond laser to treat target tissue in the vitreous cavity of an eye.

### BACKGROUND OF THE INVENTION

The vitreous body (also known as vitreous humor) is a colorless, gelatinous material that fills the vitreous cavity of an eye between the lens and the fundus. At the back of the eye, the vitreous contacts the retina and keeps the retina in place by pressing the retina against the choroid. Although the vitreous body is produced by retinal cells, it contains very few cells and is mostly water (greater than 98%). Still, despite its high water content, the vitreous includes enough collagen (vitrosin) to give the vitreous body a viscosity that is substantially greater than water (i.e. over twice as viscous as water). One important aspect of the vitreous body is that it is almost completely stagnant (i.e. is not replenished by the body to any significant degree). Because of its stagnant nature, blood, cells, byproducts of inflammation, liquefied vitreous, debris and objects which enter or develop within the vitreous cavity remain in the vitreous cavity unless surgically removed.

A vitrectomy is an ocular surgical procedure that is designed to remove some or all of the vitreous body and/or other material present in the vitreous cavity of a patient's eye. A vitrectomy may be indicated as a treatment (or part of a treatment) for abnormal eye conditions such as retinal detachment, vitreous hemorrhage (bleeding into the vitreous body), retinopathy or foreign body penetration. In a conventional vitrectomy, an ophthalmologist makes one or more scleral incisions that are sized to allow instruments to be passed through the sclera and into the vitreous cavity. These instruments can include a mechanical instrument for cutting and pulling the vitreous body out of the eye. In addition, an infusion tube may be passed through the sclera to introduce a saline-based solution into the vitreous cavity. Typically, the replacement fluid is administered at the same rate that the vitreous body is removed to maintain pressure and stability within the eye. Although these conventional methods may still have some utility, the imprecision of the mechanical cutting tool limits their use.

With the above in mind, one procedure that requires greater surgical accuracy than is provided by a mechanical cutting tool is the treatment of a vitreo-retinal traction. In more detail, as a patient ages, the vitreous body can decompose from a gel to a liquid. During this process, the vitreous mass gradually shrinks and collapses, separating and falling away from the retina. In some cases, the vitreous body simply detaches from the retina as it shrinks. In other, more serious cases, the vitreous body firmly attaches to the retina and pulls on the retina. This traction, if left unabated, can cause retinal detachment and/or retinoschisis (the splitting of the retina's neurosensory layers). In addition, the shrinking of the vitreous body can tear the central retina and cause a macular hole to form. Unfortunately, the effect of these macular holes on a patient's eyesight can be rather severe.

Floaters, as they are often called, can also be caused by changes in the vitreous body. In general, floaters are pieces of material which drift around in the vitreous cavity. In some cases, vitreous floaters can be caused by posterior vitreous detachments. Floaters can also be caused by cataract surgery, progressive liquefaction of the vitreous with age, and other mechanisms. Oftentimes, floaters consist of undissolved gelatin that floats around in the more liquid center of the vitreous. Vitreous floaters are often large enough to be seen within a patient's field of vision and often appear to the patient as a spot or spec that floats or drifts about somewhat randomly in their field of view. In some instances, depending on the number, size, and positions of the floaters within the vitreous cavity, the floaters do not substantially impair a patients vision (i.e. some floaters are tolerable by the patient without intervention). On the other hand, some floaters can substantially affect a patient's vision.

Heretofore, treatment techniques for removing floaters have included vitrectomy and laser treatment using conventional lasers designed for glaucoma treatments and posterior capsular opacification (PCO) treatments. These conventional lasers are relatively high energy lasers having a large ablation area per base pulse. The large ablation footprint increases the risk of collateral damage to non-target tissue. Because of the invasiveness, risks and potential side effects associated with the current treatment techniques, many surgeons consider the risks of these procedures to be too severe to justify treatment, in all but the most severe cases.

In addition to adversely affecting a patient's vision, floaters can affect the accuracy of a surgical laser beam during procedures in which a laser beam must pass through a floater in route to a targeted treatment area. These affected treatment areas can include posterior portions of the vitreous cavity and the retina.

In light of the above, it is an object of the present invention to provide a system and method for accurately emulsifying microscopic amounts of targeted tissue in the vitreous cavity of a patient's eye. Another object of the present invention is to provide a closed loop feedback control system and corresponding method for emulsifying targeted tissue in the vitreous cavity of the eye. Yet another object of the present invention is to provide a system and method for safely and accurately emulsifying target vitreous body tissue at a boundary between the vitreous body and an adjacent anatomical structure, such as the retina or lens, by Laser Induced Optical Breakdown (LIOB) to thereby avoid damaging the adjacent anatomical structure. Another object of the present invention is to provide a system and method for reducing the influence of vitreous floaters on a patient's vision. Yet another object of the present invention is to provide a system and method for reducing the optical impact of vitreous floaters on surgical and diagnostic laser beams that are used to treat or analyze tissue in the posterior portions of a patient's eye. Still another object of the present invention is to provide a system and method for treating target tissue in the vitreous cavity that are easy to use, are simple to implement and are comparatively cost effective.

### SUMMARY OF THE INVENTION

In accordance with the present invention, a system and method are provided for the in vivo treatment, e.g. by dissection and/or emulsification, of targeted tissue in the vitreous cavity of an eye. For example, the targeted tissue may be tissue at a boundary between the vitreous body and the retina or lens. Alternatively, the targeted tissue may be a band of vitreous body tissue that is in traction with the retina. As another example, the targeted vitreous body tissue may include tissue in the vitreous cavity that adversely affects the optical characteristics of the vitreous body due to the presence of blood, debris, floaters, a clump of collagen fiber, etc.

In operation, targeted tissue in the vitreous cavity is treated by using a laser to perform LIOB in the target tissue. In some cases, this can be followed by aspiration of an emulsification product from the vitreous cavity. In some cases, LIOB can be used to dissect tissue followed by aspiration of the dissected tissue. Thus, for the present invention, LIOB and aspiration can be employed to treat and remove tissue from the vitreous cavity rather than using a mechanical cutting instrument, as described above. To achieve the accuracy necessary for the procedures described herein, the treatment/emulsification of tissue is performed by a computer controlled laser, wherein the control reference is preferably provided by an imaging detector using a technique such as Optical Coherence Tomography (OCT).

Structurally, the system includes a laser unit for generating and directing a pulsed, femtosecond laser beam along a laser beam path to a focal spot. For this purpose, the laser unit can include one or more optics for laser beam focusing and a beam steering assembly for moving the focal spot of the laser to a selected location or along a selected path to treat a volume of target tissue. The system also includes a detector that is used for creating a three dimensional image of target tissue and its surroundings, e.g. the vitreous body (and in some cases the lens or retina). Preferably, as indicated above, this detector is an Optical Coherence Tomography (OCT) device of a type that is well known in the pertinent art for the intended purpose. Alternatively, or in addition to the OCT device, the detector can include a Scheimpflug device, confocal imaging device, optical range-finding device, ultrasound device and/or two-photon imaging device. Further, the system includes a computer that is interconnected to both the laser unit and the detector.

In some aspects of the present invention, a computer program defines a focal spot path through the targeted tissue in the vitreous cavity. Specifically, this computer program includes information about the location and dimensions of the targeted tissue requiring treatment/emulsification. Further, the computer program establishes a focal spot path through the tissue that will result in the treatment/emulsification of the targeted tissue. During the treatment/emulsification of targeted tissue in the vitreous cavity, a comparator that is connected to the detector and to the computer uses information from the computer program to determine whether there is an actual operational deviation of the focal spot from the defined focal spot path. If so, an error signal that is indicative of the deviation is generated, and the focal spot of the laser beam is moved to minimize the error signal, for example using the beam steering component of the laser unit. More specifically, the movement of the laser beam focal spot can be computer-controlled and operated with closed loop feedback. In this manner, the focal spot of the laser beam is guided using closed loop feedback by the computer program to treat/emulsify targeted tissue in the vitreous cavity of an eye by Laser Induced Optical Breakdown (LIOB).

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of a system in accordance with the present invention shown in its operational relationship with an eye, which is shown in cross section;

FIG. 2 is a cross section of an eye showing the vitreous cavity and adjacent anatomical structures;

FIG. 3A is an enlarged view of a portion of the retina and vitreous cavity of an eye, as shown surrounded by the line 3-3 in FIG. 2 illustrating a laser focal spot path for emulsifying tissue in the vitreous cavity near the retina;

FIG. 3B is an enlarged view of a portion of the retina and vitreous cavity of an eye, as shown surrounded by the line 3-3 in FIG. 2 illustrating a second laser focal spot path for emulsifying tissue in the vitreous cavity near the retina;

FIG. 3C is an enlarged view of a portion of the retina and vitreous cavity of an eye, as shown surrounded by the line 3-3 in FIG. 2 illustrating a laser focal spot path for emulsifying a band of tissue in the vitreous cavity in traction with the retina;

FIG. 4 is an enlarged view of a portion of the retina and vitreous cavity of an eye, as shown surrounded by the line 4-4 in FIG. 2 illustrating a laser focal spot path for emulsifying tissue in the vitreous cavity that has been optically degraded by the presence of a floater, blood or foreign matter; and

FIG. 5 is a functional block diagram of a closed-loop control system incorporating components of the present invention.

### DESCRIPTION OF THE PREFERRED EMBODIMENTS

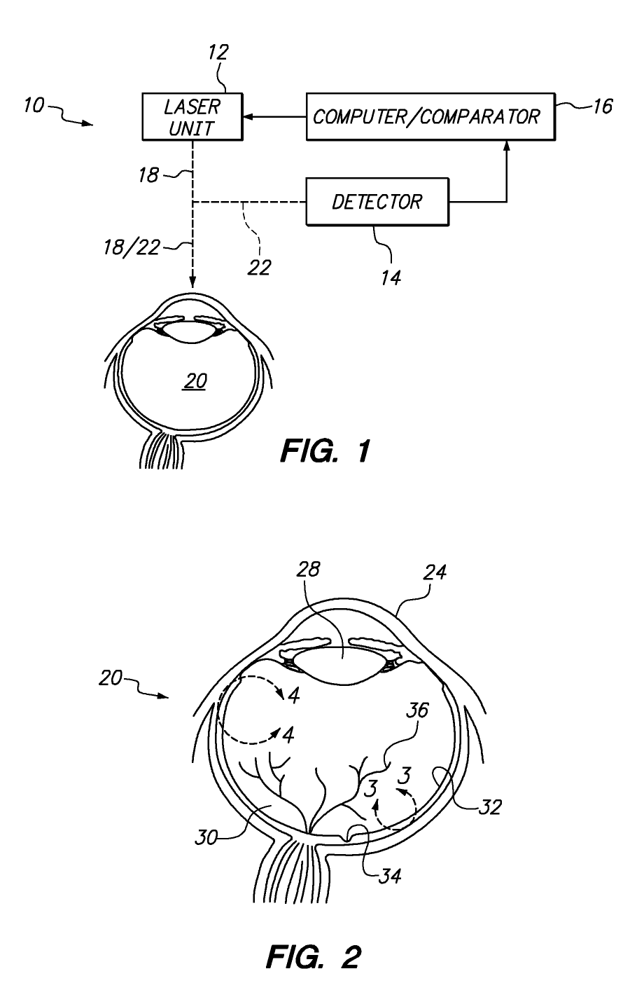

Referring initially to FIG. 1, a system for treating targeted tissue in the vitreous cavity of an eye is shown and is generally designated 10. As shown, the system 10 includes a laser unit 12, a detector 14 and a computer/comparator 16. In the system 10, the detector 14 is operationally connected to the computer/comparator 16, and the computer/comparator 16 is connected directly to the laser unit 12. With this combination, the system 10 is used to generate and direct a laser beam 18 toward an eye 20 for an ophthalmic surgical procedure as envisioned for the present invention.

For the purposes of the present invention, the laser unit 12 is capable of generating a so-called “femtosecond” laser beam 18. Thus, the generated laser beam 18 includes a sequence of laser pulses having a very ultra-short duration (e.g. less than approximately 500 fs). In addition, the laser unit 12 can include a beam steering component for moving the focal spot of the laser along a selected path to emulsify a volume of target tissue. For example, the beam steering component can include a pair of mirrors (not shown) mounted on respective tip-tilt actuators to steer the beam in respective, orthogonal directions. Importantly, the laser beam 18 must be capable of performing Laser Induced Optical Breakdown (LIOB) on selected target tissue inside the eye 20. Further, it is important for there to be a precise performance of this LIOB. Such precision requires there be a capability of imaging the target tissue that is to be altered by LIOB.

The detector 14 is preferably a type of imaging unit that operates using Optical Coherence Tomography (OCT) techniques. Alternatively, or in addition to the OCT device, the detector 14 can include a Scheimpflug device, confocal imaging device, optical range-finding device, ultrasound device and/or two-photon imaging device. Thus, the detector 14 will include a light source to generate an imaging beam 22 and optics to direct the imaging beam 22 toward the eye 20. In some cases, these optics can include some or all of the optics in the beam steering component of the laser unit 12. For the system 10, the imaging beam 22 is used to create three dimensional images of selected tissues within the eye 20. As indicated in FIG. 1, these images are then passed to the computer/comparator 16 for use by the computer/comparator 16 in controlling the laser unit 12. As envisioned for the present invention, the precision required for this control will be best appreciated with reference to FIG. 2.

FIG. 2 identifies several pertinent structures in the eye 20 including the cornea 24, the sclera 26, the lens 28, vitreous body 30, retina 32, macula 34 and retinal blood vessels 36. As shown the vitreous body 30 resides in the vitreous cavity which extends from the retina 32 and macula 34, posteriorly, to the lens 28, anteriorly. As such, the vitreous body 30 establishes borders with the lens capsule, retina 32, macula 34 and retinal blood vessels 36.

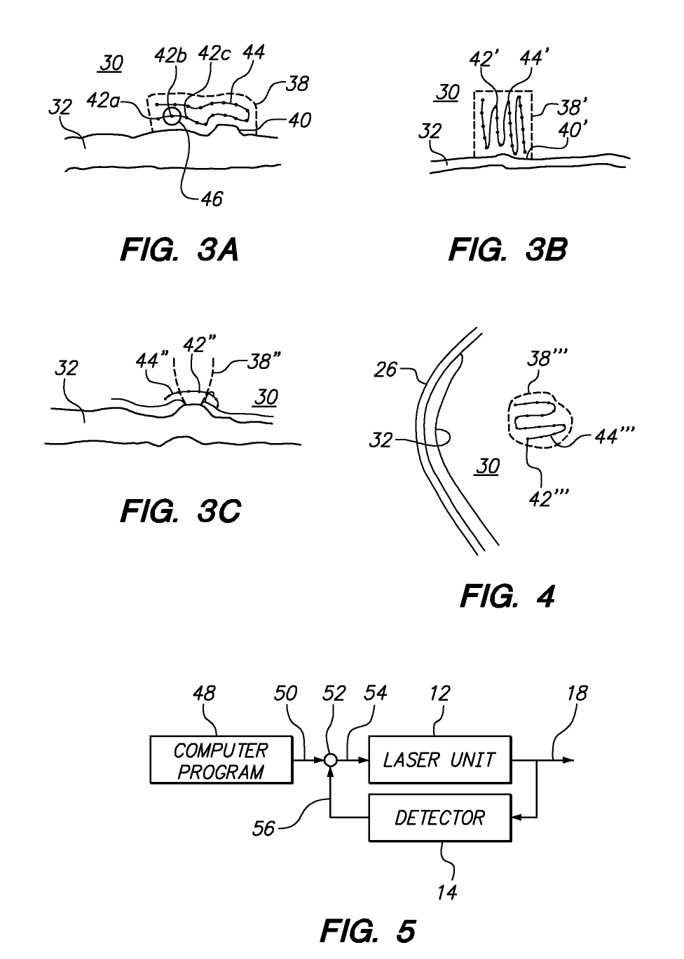

Several situations are of particular interest for the present invention. For one, as shown in FIG. 3A, there is interest in accurately emulsifying target vitreous body tissue 38 at a boundary 40 between the vitreous body 30 and an adjacent anatomical structure, such as the retina 32. It is to be appreciated that the current discussion is equally applicable to other vitreous body boundaries including boundaries with the lens capsule, retinal blood vessels 36, the macule 34, etc. FIG. 3A shows a plurality of focal spot positions, of which focal spot positions 42a-c are labeled. Together, the focal spot positions 42a-c define a focal spot path 44 that can be followed by the laser beam 18 (see FIG. 1) to emulsify target vitreous body tissue 38.

FIG. 3A illustrates an amount of tissue (Circle 46) that is emulsified at a single focal spot position, for example, by LIOB upon irradiation by one or more laser pulses. In accordance with the present invention, a computer program can be used to define the focal spot path 44 through the targeted vitreous body tissue 38 that will result in the emulsification of the targeted vitreous body tissue 38. Specifically, this computer program can include information about the location and dimensions of the targeted vitreous body tissue 38 requiring emulsification.

Cross-referencing FIGS. 1 and 3A, it can be appreciated the focal spot path 44 can be followed by the laser beam 18 (see FIG. 1) to emulsify target vitreous body tissue 38 using closed loop feedback control. Specifically, the computer/comparator 16 can include a computer program that defines a reference input for the system 10 including the desired focal spot path 44 for emulsifying targeted vitreous body tissue 38 that will result in the emulsification of the targeted vitreous body tissue 38. Moreover, the laser unit 12 is responsive to an actuating signal from the computer/comparator 16 to establish an output for directing a laser pulse from the laser unit 12 to a focal spot within the targeted vitreous body tissue 38 to emulsify targeted vitreous body tissue 38 by Laser Induced Optical Breakdown (LIOB). For the system 10, the detector 14 creates an image of the targeted vitreous body tissue 38 after the pulse. Next, the computer/comparator 16 receives the output from the laser unit 12 and the image from the detector 14, to generate a feedback error signal based on the reference input. In more quantitative terms, the feedback error signal is a measure of a deviation of the pulse location imaged by the detector 14 from a corresponding desired focal spot location in the reference input. As described in more detail below with reference to FIG. 5, the feedback error signal can then be used for modifying the actuating signal to the laser unit 12 to minimize the feedback error signal for subsequent laser pulses.

FIG. 3B illustrates the focal spot positions 42′ that define another focal spot path 44′ that can be followed by the laser beam 18 (see FIG. 1) to emulsify target vitreous body tissue 38′ at or near boundary 40′ between the vitreous body 30 and an adjacent anatomical structure, such as the retina 32. For the focal spot path 44′ shown in FIG. 3B a series of focal point scans are included in which the laser beam 18 approaches the boundary 40′ while image updates allow for the emulsification of target vitreous body tissue 38′ at or very close to the boundary 40′. In accordance with the present invention, a computer program can be used to define the focal spot path 44′ through the targeted vitreous body tissue 38′ that will result in the emulsification of the targeted vitreous body tissue 38′. Specifically, this computer program can include information about the location and dimensions of the targeted vitreous body tissue 38′ requiring emulsification.

FIG. 3C shows another situation of particular interest for the present invention. As shown, and discussed previously above, there is interest in accurately emulsifying a band of target vitreous body tissue 38″ that is in traction with the retina 32. FIG. 3C shows a plurality of focal spot positions 42″, which together, define a focal spot path 44″ that can be followed by the laser beam 18 (see FIG. 1) to emulsify target vitreous body tissue 38a″ to sever the target vitreous body tissue 38″ in traction with the retina 32. In accordance with the present invention, a computer program can be used to define the focal spot path 44″ through the targeted vitreous body tissue 38″ that will result in the emulsification of the targeted vitreous body tissue 38″. Specifically, this computer program can include information about the location and dimensions of the targeted vitreous body tissue 38″ requiring emulsification.

FIG. 4 shows another application in which the system 10 shown in FIG. 1 can be used to emulsifying target tissue 38′″ in the vitreous cavity. Specifically, FIG. 4 illustrates that the target tissue 38′″ can be located anywhere in the vitreous cavity. For example, the target tissue 38′″ may include tissue such as floaters in the vitreous cavity that adversely affect the optical characteristics of the eye. Alternatively, the target tissue 38′″ may include tissue to be removed from the vitreous cavity due to the presence of blood, debris a clump of collagen fiber, etc. In some applications, the system 10 described herein can be used to treat neo-vascular membranes.

FIG. 4 shows a plurality of focal spot positions 42′″, which together, define a focal spot path 44′″ that can be followed by the laser beam 18 (see FIG. 1) to emulsify target tissue 38′″ within the vitreous cavity. In accordance with the present invention, a computer program can be used to define the focal spot path 44′″ through the targeted tissue 38′″ that will result in the emulsification of the targeted tissue 38′″. Specifically, this computer program can include information about the location and dimensions of the targeted tissue 38′″ requiring emulsification. Optical defects such as floaters may also be treated using a femtosecond laser by using one or more laser pulses to break a floater into smaller fragments. In this process, some or all of the fragments or the entire floater may be projected to a more peripheral portion of the eye rendering them less harmful to a patient's vision or a laser surgical procedure. For example, the laser beam may be directed to a location adjacent the floater to project the floater to the periphery. Thus, using a femtosecond laser, treatment of floaters can be accomplished by dissolution (ablation), by fragmentation and displacement of fragments or the entire floater toward the eye's periphery, or by rejuvenation of the vitreous body (e.g. homogenization of hitherto liquid and gel phases). Updated images from the detector 14 (FIG. 5) can be used to track the moving floaters and/or the projected floaters/fragments. Closed loop operation may be used to position the focal point on or adjacent to the floaters/fragments as the floaters/fragments move.

Laser treatment of floaters and other tissue that adversely affect the optical characteristics of the eye can be treated to improve a patient's vision and/or to remove the floater/substance to allow a surgical laser to more accurately treat tissue at a more posterior location within the eye. Use of a femtosecond laser as described herein allows for less collateral damage to non-target tissue than a conventional high energy laser such as the conventional lasers used for glaucoma or PCO treatment. This allows for floaters closer to a delicate anatomical feature such as the retina or lens to be treated.

Use of a computer controlled femtosecond laser with imaging feedback as described herein also allows for more precise z-targeting (where z is an axis in the direction of beam propagation) as compared with some floater treatment techniques in which the z-position of the laser spot is set manually by the surgeon by overlapping two laser spots. The use of a computer controlled femtosecond laser with imaging feedback as described herein can also result in a substantial reduction in floater treatment procedure time from the current treatment time of about 20-30 minutes to, less than about a minute in some cases.

The use of a computer controlled femtosecond laser with imaging feedback using detectors such as OCT, etc., as described herein, can also provide a diagnostic means to allow better patient selection for treatment of floaters and other localized tissue abnormalities. This can allow the surgeon to better distinguish between patients who will benefit from laser surgery from those who are merely ‘obsessive-compulsive’ and can also better identify the position and size of floaters with respect delicate ocular structures such as the lens and retina. Accurate images using the image techniques described herein can be used to define non-treatment safety zones to protect the lens, posterior lens capsule, retina, etc.

In many instances, it may be advantageous to combine floater treatment with treatment of the lens (cataract) or retina in an integrated surgical procedure. Due to the mobile nature of floaters, it may be desirable to track and/or treat a floater just prior to a more posterior laser procedure. This can be accomplished using the imaging techniques described herein. In addition, with the use of a computer controlled femtosecond laser with imaging feedback, as described herein, both procedures may be performed with the same equipment. In addition, it may be advantageous to remove floaters prior to retinal surgery or other posterior eye surgery because floaters can degrade the quality of beams aimed at the retina/posterior portions of the eye. Beam quality can also be increased by using a wavefront detector capable of measuring wavefront distortions in a laser beam during propagation in the eye together with an adaptive mirror operable on the laser beam to offset the wavefront distortions. Removal of floaters in combination with a wavefront detector adaptive mirror can ensure the high beam quality necessary to perform many surgical procedures on the retina/posterior portions of the eye.

FIG. 5 indicates that the system 10 may be computer-controlled and operated with closed loop feedback. For this operation, a computer program 48 is provided for use with the computer/comparator 16 (See FIG. 1). Specifically, the computer program 48 will include a definition for the focal spot paths 44, 44′, 44″, 44′″. This definition will necessarily include the location and the dimensions of each target vitreous body tissue 38, 38′, 38″ 38′″. In order to establish a location for the target vitreous body tissue 38, 38′, 38″ 38′″, as well as for other laser functions, the computer program 48 provides a reference input 50 for the system 10.

In the operation of system 10, the reference input 50 from the computer/comparator 16 (i.e. computer program 48) is sent to a summing point 52. It is then sent from the summing point 52 as an actuating signal 54 for the laser unit 12 including the beam steering component of the laser unit. Thus, the laser beam 18 is generated as an output from the laser unit 12 in accordance with the actuating signal 54. For guidance and control purposes, the output of the laser unit 12 (i.e. laser beam 18) is monitored by the detector 14. Further, the detector 14 creates three dimensional images that show the effects of LIOB in the target vitreous body tissue 38, 38′, 38″ 38′″. These images are then used as the basis for generating feedback (error) signals 56 that are returned to the summing point 52. At the summing point 52, the reference input 50 (i.e. definition of focal spot path) is compared with the feedback (error) signal 56 (i.e. images from the target vitreous body tissue 38, 38′, 38″ 38′″). This comparison is then used to appropriately adjust the actuating signal 54. As with any closed loop feedback control system, the objective here is to maintain the feedback (error) signal 56 at a null.

While the particular Systems and Methods for Treating Target Tissue in the Vitreous Cavity as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for in vivo treatment of target tissue in the vitreous cavity of the eye, the system comprising:
a laser unit for generating a laser beam, and for directing the laser beam along a laser beam path to a focal spot to treat target tissue by Laser Induced Optical Breakdown (LIOB);
a detector for creating an image of the target tissue;
a computer connected to the laser unit, and to the detector, for guiding the focal spot of the laser beam in accordance with a predetermined computer program, wherein the computer program defines a focal spot path, through the target tissue within the vitreous cavity; and
a comparator connected to the detector and to the computer for determining a deviation of the focal spot from the defined focal spot path to produce an error signal indicative of the deviation, and for moving the focal spot to minimize the error signal during treatment of the target tissue.

2. A system as recited in claim 1 wherein the laser beam is a pulsed femtosecond laser beam, and the detector is selected from the group consisting of an Optical Coherence Tomography (OCT) device, a Scheimpflug device, a confocal imaging device, a optical range-finding device, an ultrasound device and a two-photon imaging device.

3. A system as recited in claim 1 wherein the eye has a retina and the target tissue is in the vitreous cavity adjacent to the retina.

4. A system as recited in claim 1 wherein the eye has a retina and the target tissue is a band of vitreous body tissue in traction with the retina.

5. A system as recited in claim 1 further comprising an aspirator for removing vitreous body from the eye.

6. A system as recited in claim 1 wherein the focal spot has a waist that is less than 15 μm in diameter.

7. A system as recited in claim 1 wherein the laser unit delivers a train of laser pulses and each pulse has a pulse energy that is large enough to emulsify the target tissue by laser-induced optical breakdown (LIOB).

8. A system as recited in claim 1 wherein the laser unit delivers a train of laser pulses including a first pulse and a second pulse and wherein the detector creates an image of the target tissue between the first pulse and the second pulse.

9. A system as recited in claim 1 wherein the target tissue is a vitreous floater.

10. A system as recited in claim 1 wherein the treatment dissects tissue.

11. A system as recited in claim 1 wherein the treatment emulsifies tissue.

12. A closed loop feedback control system for emulsifying target tissue in the vitreous cavity of the eye which comprises:
a computer with a computer program, wherein the computer program defines a reference input for the system including a desired focal spot path for emulsifying target tissue in the vitreous cavity;
a laser unit for generating a pulsed laser beam, wherein the laser unit is responsive to an actuating signal from the computer to establish an output for directing a laser pulse from the laser unit to a focal spot within the target tissue to emulsify tissue by Laser Induced Optical Breakdown (LIOB);
a detector for creating an image of the target tissue after the pulse; and
a comparator for receiving the output from the laser unit, and for receiving the image from the detector, to generate a feedback error signal based on the reference input, wherein the feedback error signal is a measure of a deviation of the pulse location imaged by the detector from a corresponding desired focal spot location in the reference input, and wherein the feedback error signal is used for modifying the actuating signal to the laser unit to minimize the feedback error signal.

13. A system as recited in claim 12 wherein the detector is selected from the group consisting of an Optical Coherence Tomography (OCT) device, a Scheimpflug device, a confocal imaging device, a optical range-finding device, an ultrasound device and a two-photon imaging device.

14. A system as recited in claim 12 wherein the laser beam is a pulsed femtosecond laser beam.

15. A system as recited in claim 12 wherein the eye has a retina and the target tissue is in the vitreous cavity adjacent to the retina.

16. A system as recited in claim 12 wherein the eye has a retina and the target tissue is a band of vitreous body that is in traction with the retina.

17. A system as recited in claim 12 further comprising an aspirator for removing emulsified vitreous body from the eye.

18. A method for emulsifying target tissue in the vitreous cavity of an eye which comprises the steps of:
generating a laser beam;
directing the laser beam along a laser beam path to a focal spot in the target tissue;
guiding the focal spot of the laser beam in accordance with a predetermined computer program to emulsify tissue by Laser Induced Optical Breakdown (LIOB), wherein the computer program defines a focal spot path for emulsifying target tissue;
creating an image of the target tissue;
determining a deviation of the focal spot from the image of the focal spot path;
producing an error signal indicative of the deviation; and
moving the focal spot of the laser beam to minimize the error signal during emulsification of the target tissue.

19. A method as recited in claim 18 wherein the laser beam is a pulsed femtosecond laser beam.

20. A method as recited in claim 18 wherein the creating step is accomplished by a device selected from the group consisting of an Optical Coherence Tomography (OCT) device, a Scheimpflug device, a confocal imaging device, a optical range-finding device, an ultrasound device and a two-photon imaging device.

21. A method as recited in claim 18 wherein the performance of the method is controlled by a computer.

22. A method as recited in claim 18 wherein the eye has a retina and the target tissue is a band of vitreous body that is in traction with the retina.

23. A method as recited in claim 18 wherein the target tissue is a vitreous floater.

24. A method as recited in claim 23 wherein the vitreous floater is positioned at a floater location and further comprising the step of treating tissue posterior to the floater location with a laser beam in an integrated surgical procedure with the treatment of the floater.

25. A method as recited in claim 24 wherein the laser unit comprises a wavefront detector for measuring wavefront distortions in said laser beam during propagation in said eye and an adaptive mirror operable on said laser beam to offset said wavefront distortions.

26. A method as recited in claim 23 wherein the vitreous floater is treated by dissolution.

27. A method as recited in claim 23 wherein the vitreous floater is treated by breaking the vitreous floater into pieces.

28. A method as recited in claim 23 further comprising the step of treating retinal tissue with a laser beam in an integrated surgical procedure with the treatment of the floater.

29. A method as recited in claim 23 further comprising the step of treating cataract with a laser beam in an integrated surgical procedure with the treatment of the floater.

30. A computer program product comprising program sections for respectively:
defining a focal spot path to emulsify target tissue in the vitreous cavity of an eye;
creating an image of the focal spot path;
directing a laser beam along a laser beam path to a focal spot in the target tissue;
guiding the focal spot to alter material by Laser Induced Optical Breakdown (LIOB) to emulsify the target tissue;
determining a deviation of the focal spot from the image of the focal spot path;
producing an error signal indicative of the deviation; and
moving the focal spot of the laser beam to minimize the error signal during emulsification of the target tissue.
